(19) United States
(12) Patent Application Publication
(10) Pub. No.: US 2011/0306058 A1
(43) Pub. Date: Dec. 15, 2011

(54) METHOD FOR THE FUNCTIONAL MEASUREMENT OF PLASMATIC THROMBODULINE ACTIVITY

(75) Inventors: Patrick Van Dreden, Brennilis (FR); Aurelie Rousseau, Louveciennes (FR)

(73) Assignee: DIAGNOSTICA STAGO, Asnieres (FR)

(21) Appl. No.: 12/516,895

(22) PCT Filed: Nov. 28, 2007
(86) PCT No.: PCT/FR2007/001955

§ 371 Date: May 29, 2009

(30) Foreign Application Priority Data
Nov. 29, 2006 (FR) 0610444

(51) Int. Cl. G01N 33/573

(52) U.S. Cl. 435/7.4

(57) ABSTRACT

A method for the in vitro measurement of the thromboduline functional activity, includes dosing in a biological medium, and from a biological sample, the thrombine-activation of C protein into activated C protein (PCa) in the presence of its co-factor or the thromboduline, the method including adding to the sample plasma the agents necessary for activating the C protein system, adding purified C protein and also adding a fibrin polymerisation inhibitor. Also described is application of the method in the detection of coagulation pathologies.

The invention relates to the field of hemostasis and in particular to coagulation disorders.

The present application pertains to a method for measuring the functional activity of plasmatic thrombomodulin in a biological sample.

The method of the invention is carried out in vivo using a biological sample, in particular a blood sample taken from a patient.

The method of the invention allows the functional activity of thrombomodulin in a plasmatic medium to be measured.

In particular, the present application envisages a method for determining the functional activity of thrombomodulin in a plasmatic medium, said determination being carried out, for example, using a chromogenic or fluorometric method.

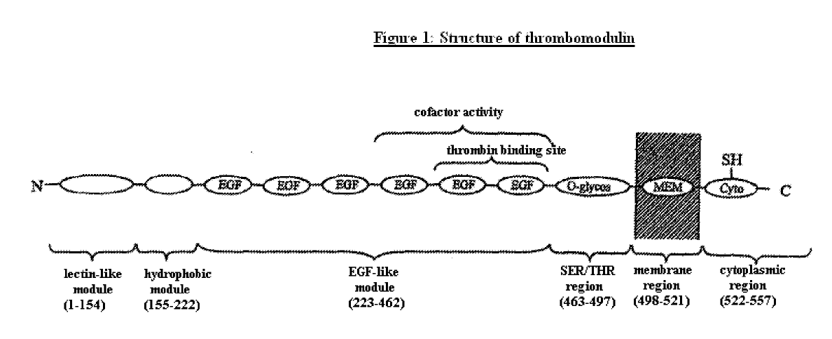

Thrombomodulin (TM) is a thrombin receptor proteoglycan forming an integral part of the membrane of endothelial cells. Responsible in part for the anticoagulant properties of the endothelium, it is a 557 amino acid protein which is in three portions: a C-terminal intra cytoplasmic portion involved in endocytosis mechanisms; a hydrophobic intra-membrane portion; and a N-terminal portion constituted by a long peptide chain. This latter comprises four regions: a Ser/Thr rich region, followed by a region of six domains which are homologues of epidermal growth factor (EGF-like), a hydrophobic region and finally a lectin-like region (FIG. 1).

In 1981, Owen W. G. and Esmon C T characterized the functional activity of thrombomodulin (Proceedings of the National Academy of Science USA, 1981, vol. 78, pages 2249-52 and Journal of Biological Chemistry 1981 vol 256, pages 6632-5), demonstrating activation of plasmatic protein C to activated anticoagulant protein C, by thrombin, catalyzed by thrombomodulin.

The coding sequence for human thrombomodulin was characterized and identified by Wen D Z et al in 1987 (Biochemistry, 1987, vol 26, pages 4350-7) in EMBL under reference number BC 035602 and Weiler H. et al (J. Thromb. Haemost. 2003 July, 1(7): 151-24).

TM is present on the surface of all blood vessels (arteries, veins, capillaries) and lymphatic vessels of the organism, with a particular abundance in richly vascularized organs such as the placenta and lung. It is also expressed by keratinocytes, osteoblasts, mesangial cells, platelets and neutrophils.

The regulation of TM synthesis is principally under the dependency of cyclic AMP, TNFα, IL-1 and retinoic acid.

TM is not secreted by endothelial cells and is not degraded by thrombin. Only under the effect of different types of attacks of the endothelial cell (elastase from polynuclears; cytokines, macrophages, lipopolysaccharides, etc) can endothelial TM be cleaved into fragments which are liberated into the circulation in the soluble form then eliminated renally. Once cleaved, cellular TM is in the soluble, i.e. plasmatic, form of TM, having had the cytoplasmic and mesoporous regions deleted (Ishiih et al, 1985, J Clin Invest 76, 2178). Assaying soluble plasmatic TM (TMp) constitutes a good marker for endothelial lesion.

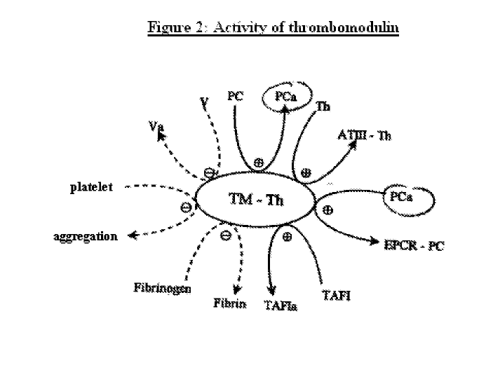

The principal function of TM is to oppose the procoagulant action of thrombin by various mechanisms. When binding with TM, thrombin (Th) undergoes a conformational modification. It loses its coagulant properties and its affinity for platelets and for fibrinogen. However, it can still be inactivated by antithrombin III (ATIII). Formation of the TM-Th complex is decoupled in the presence of chondroitin sulfate. These interactions allow TM to eliminate Th circulating in the plasma. This TM-Th complex is capable of activating protein C which, in the presence of S protein, inhibits factors Va and VIIIa; this succession of interactions and reactions constitutes the protein C system. The TM-Th complex also plays a role in the activation of protein C bound to endothelial protein C receptor (EPCR) as well as the activation of a plasmatic procarboxypeptidase involved in fibrinolysis: TAFI (thrombin activatable fibrinolysis inhibitor). Finally, TM has an antithrombotic activity in the presence of ATIII. A role of TM in inflammation and in cellular proliferation/differentiation has also been evoked (FIG. 2).

Thus, thrombomodulin acts as a coagulation inhibitor, in particular by accelerating the activation of protein C into activated protein C (PCa) which itself, in the presence of protein S cofactor, inactivates the coagulation factors Va and VIIIa.

The in vivo synthesis of thrombomodulin is augmented by various factors, such as AMPc, retinoids, thyroid hormones, and hyperthermia (Conway E M et al, 1994, J Biol Chem, 269, 22804). Other agents such as cytokines, IL-1, TNF, LPS, and homocysteine as well as intracellular micro-organisms (cytomegalovirus, herpes, rickettsies) have a negative influence on its expression and its activity, thus leading to a transitory increase in the procoagulant activity of endothelial cells.

While the levels of soluble TM (TMs) are high in newborns, age has no influence in healthy subjects whether they are of 20 or 60 years of age, but the sex of an individual has an influence as males have been observed to have significantly higher values than females.

A rise in the TMs level associated with an attack of the endothelial has been described in numerous pathologies such as DIVC, diabetes, chronic myeloid leukemia, hepatic and renal insufficiencies, but also in preeclampsia, thrombotic thrombocytopenic purpura, rickettsioses, Behcet's disease, homocystinuria. Reduced levels have been observed in studies on patients with pulmonary arterial hypertension and during the course of melanoma. Finally, mutations in the gene for TM have been described and associated with a reduction in the level of TMs.

On a therapeutic level, the perfusion of recombinant TM in animals has proved effective in preventing the occurrence of thrombosis.

Because of its direct influence on the steps of coagulation, in particular in the activation of the protein C system, it appears to be of interest to be able to determine the functional activity of thrombomodulin.

Tests for determining the activity of thrombomodulin have already been proposed in the prior art, but they do not result in a determination of the activity of thrombomodulin in plasmatic systems, only in cellular systems. Recently, a plasmatic medium assay method was described by Öhlin A K et al (Journal of Thrombosis and Hemostasis, 2005, 3: 976-982). However, this publication does not propose a direct test of TMs but necessitates a first step for isolation and separation using monoclonal antibodies directed against plasma thrombomodulin. In fact, in a first step of the test described by Ohlin A K et al, the test plasma, which may be diluted, is incubated on a microplate the wells of which comprise a monoclonal antibody against human thrombomodulin. After removing the plasma, the wells of the microplates are washed to retain only the fragments of thrombomodulin which have bound the antibodies. In a second step, the wells of the prepared microplate are brought into the presence of the reagents necessary to obtain activated protein C. After incubation, a chromogenic or fluorometric substrate can reveal the activated protein C.

The authors of the publication concluded that it was important in future to design a test which could directly measure in plasma the activity of thrombomodulin without passing through a step for isolation and separation of the thrombomodulin.

Thus, currently, assaying plasmatic thrombomodulin resides solely in antigenic assay of soluble protein which corresponds to the cleaved membrane protein which has lost the carboxy-terminal cytoplasmic and trans-membrane hydrophobic region. TMs remains capable of binding thrombin and activating protein C, which are functions which are localized on the repeated EGF motifs.

Various tests using immunological reactions between anti thrombomodulin antibodies and soluble thrombomodulin are currently available. Diagnostica Stago, for example, offers an ELISA test to assay thrombomodulin (Aserachrom® Thrombomodulin).

The problem with in vitro assaying of the functional activity of thrombomodulin is that which the invention proposes to overcome by proposing a method for measuring the functional activity of thrombomodulin in a biological medium and in particular a plasmatic medium, i.e. a method which can allow in vitro assay of soluble thrombomodulin contained in the biological medium containing it, in particular in plasma, without separating the thrombomodulin from the other constituents of the test biological medium, in particular plasma.

In other words, the invention proposes a test for measuring thrombomodulin in a biological medium, in particular in a plasmatic medium, in the presence of the reagents necessary for activation of the protein C system. Further, the invention proposes a test which, in a biological medium and in particular a plasmatic medium, can overcome the problems linked to coagulation of the sample under the effect of the reagents necessary for activation of protein C.

The method of the invention and the means for implementation thereof are defined for use in the context of biomedical investigations carried out in vitro.

To recapitulate, the activation of protein C to PCa occurs in vivo under the action of the complex formed on the surface of the vascular endothelium by thrombomodulin and thrombin (factor IIa). The PCa then complexes with protein S to bind itself onto phospholipids on the surface of platelets or the endothelium. These PCa-PS-phospholipid complexes catalyse inactivation of factors Va and VIIIa, thereby slowing down the coagulation phenomena.

Thus, the invention concerns a method for in vitro measurement of the functional activity of thrombomodulin, said measurement being carried out in a biological medium from a sample which has been taken from a patient. In accordance with a particular implementation of the invention, a sample which can allow the thrombomodulin activity in a biological medium to be measured is a plasma sample. In accordance with another particular implementation of the invention, the sample allowing the thrombomodulin activity to be measured is a sample of whole blood or cephalo-rachidian liquid (CRL) or amniotic liquid.

The in vitro assay method of the invention comprises measuring, in a biological sample medium, the activation of protein C to activated protein C (PCa), said activation being carried out under the effect of thrombin, in the presence of its cofactor, namely thrombomodulin from the sample. The measurement is carried out after adding purified protein C in excess. Other agents necessary for activation of the protein C system are also added to the sample, as well as a fibrin polymerization inhibitor.

In particular, the invention pertains to a method for in vitro measurement of the functional activity of plasmatic thrombomodulin, said method comprising assaying in a plasmatic medium, the activation of protein C to activated protein C (PC a) by thrombin in the presence of its cofactor, said plasmatic thrombomodulin. To carry out this measurement, excess purified protein C, termed purified exogenous protein C, is added to the test plasma, as well as the agents necessary for activation of the protein C system, and a fibrin polymerization inhibitor.

As an alternative to adding purified protein C, it is possible to add TAFI (plasmatic carboxypeptidase U, the activation of which is catalyzed by thrombin, is accreted for thrombomodulin) in order to measure its activated TAFI activation (TAFIa).

The measurement of the activity is termed a measure of the functional activity of thrombomodulin, as it allows the activity of the thrombomodulin in a biological medium to be measured, in particular in a plasmatic medium by adding to the medium, in particular the test plasma, protein C in excess and the agents necessary for activation of the protein C system. It will be observed that the excess protein C used is sufficient to overcome, during the measurement, any quantitative or qualitative deficit relating to the presence of endogenous protein C in the test medium. In other words, the term “excess exogenous protein C” means, in implementing the invention, that the exogenous protein C is added in a quantity such that the measurement of its PCa activation is independent of the amount of protein C in the sample.

In accordance with a particular implementation of the invention, the assay carried out in a plasmatic medium is carried out on a plasma sample obtained from a biological sample which has been taken from a patient.

The plasmatic thrombomodulin which is measured in accordance with the invention is soluble protein (soluble thrombomodulin or TMs, also termed plasmatic thrombomodulin, TMp) which corresponds to cleaved membrane thrombomodulin and thus separated from its cytoplasmic carboxyterminal and transmembrane hydrophobic regions. In fact, the soluble thrombomodulin remains capable of binding thrombin and activating protein C into PCa since this activation function is located on the repeat EGF motifs of the protein.

In contrast, when the activity of the thrombomodulin is measured in a LCR or amniotic liquid sample, the thrombomodulin is in its complete (non truncated) form.

The agents necessary for activation of the protein C system which are added to the test plasma sample are principally thrombin (in particular purified human α-thrombin) and calcium ions, depending on the various implementational modes of the invention.

The calcium ions are, for example, supplied in the form of CaCl₂.

Further, the method for measuring the activity of plasmatic thrombomodulin in accordance with the invention comprises adding to the test sample an inhibitor which is in competition with thrombin and thus blocks the transformation of fibrinogen into fibrin.

A method for the in vitro measurement of the functional activity of plasmatic thrombomodulin in accordance with the invention comprises:
bringing a thrombomodulin activity test plasma sample into contact with purified exogenous protein C, thrombin, calcium ions and a fibrin polymerization inhibitor;
incubating the medium obtained to allow the production of activated protein C;
functional assay of the activated protein C.

In a particular implementation of the measurement method of the invention, the quantity of activated protein C formed is assayed by its activity on a substrate. In particular, this substrate is an enzymatic substrate, which may be natural or synthetic; as an example, it may be a chromogenic or fluorometric substrate.

A suitable substrate for assaying the activity of the PCa may be a protein substrate, for example a peptide.

In a particular implementation of the invention, the enzymatic substrate of the measured activated protein C is a substrate which means that the amidolytic activity of the PCa can be assayed. In particular, it is a chromogenic or fluorometric substrate.

Suitable synthetic substrates for use in the context of the invention which may be cited are synthetic oligopeptide substrates. An example which may be mentioned is the synthetic substrate CBS 42.46 from Diagnostica Stago (oligopeptide: THC-Pro-Arg-pNa). Another example of a chromogenic substrate is the substrate S-2366 (oligopeptide pyroGlu-Pro-Arg-pNa) or S-2238 (oligopeptide H-D-Phe-Pip-Arg-pNa).

In the context of the invention, various fibrin polymerization inhibitors may be selected. Examples which may be cited are synthetic peptide inhibitors, for example the oligopeptide inhibitor H-Gly-Pro-Arg-Pro-OH.AcOH (GPRP.AcOH) (Pefabloc® FG from Pentapharm).

In a particular implementation of the invention, the method for measuring the functional activity of plasmatic thrombomodulin comprises the following steps:
a) bringing a plasma sample into contact with a reagent 1 comprising thrombin, contained in a medium which can activate the protein C system, in particular comprising calcium ions, the reagent 1 further comprising a fibrin polymerization inhibitor;
b) incubating the medium constituted in step a) with a reagent 2 comprising purified protein C, for a period which is sufficient to allow its activation to PCa by the thrombin complexed to its cofactor, namely the thrombomodulin contained in the plasma sample;
c) bringing the medium constituted in step b) comprising activated protein C into contact with an enzymatic substrate of PCa;
d) quantifying the transformation of the substrate of the PCa.

In order to implement the invention, the protein C used is a purified protein C, for example protein C isolated using the method described by Ohlin A K et al, J Biol Chem, 1988, 263: 19240-8. In accordance with a particular implementation of the invention, the protein C used is purified human protein C. The protein C used is supplied in a concentration in the range 0.5 μM/l to 2.5 μM/1, or even 0.1 to 10 μM/1.

The protein C is termed “purified” in order to show that it is substantially free of other protein Components and contaminants, independently of any reference to its mode of preparation.

The protein C supplied may be a recombinant protein.

The protein C is also available in its purified and active form for carrying out the measurement of the invention, from Diagnostica Stago, Enzyme Research Laboratories, or American Diagnostics.

In the context of the method of the invention, bringing the supplied thrombin into contact with thrombomodulin present in the test plasma results in formation of the thrombin/thrombomodulin complex.

The reagent supplying thrombin and the calcium ions may be constituted by thrombin diluted in a buffer comprising calcium ions. The fibrin polymerization inhibitor may also be added to the buffer. Other constituents which are routinely contained in this type of buffer may also be added.

The thrombin used is advantageously of human origin. In particular, it is thrombin the activity of which, expressed in NIH units, is in the range 5 to 20 NIH, advantageously 10 NIH in the reagent which is used to prepare the reaction medium.

The reagent thus constituted allows the supplied thrombin to interact with the thrombomodulin of the plasma sample and activation of the protein C.

Examples of other components of the constituted reagent which may be cited are a heparin inhibitor or another anticoagulant, used if the test sample has been taken from a patient treated with heparin or with another anticoagulant and is thus characteristic of a patient treated with heparin or another anti coagulant.

If, however, the sample derives from a patient treated with an anticoagulant from the anti-vitamin K family, no inhibitor of that anticoagulant is added since no inhibitor is currently available.

As an alternative to diluting the sample in Owren Koller buffer, dilution may be carried out in normal plasma in accordance with the usual selection criteria which are known to the skilled person, said plasma being rendered deficient in thrombomodulin. The exogenous thrombin is then added to this plasma as well as purified protein C and the fibrin polymerization inhibitor and, as indicated above, the heparin inhibitor when the biological sample comes from a patient treated with heparin, or another inhibitor if it is a different anticoagulant. The thrombin is supplied by the plasma.

The term “thrombomodulin deficient plasma” means a plasma which comprises less than 1% of this protein.

A thrombomodulin-deficient plasma may be prepared using any method which is known per se.

As an example, it is possible to use an immunoadsorption technique employing antibodies directed against this protein, for example antibodies immobilized on a support.

The plasma to be treated is citrated.

The purified protein C is supplied under conditions which allow it to react with the thrombin/thrombomodulin complex, resulting in activation of the protein C to the form of an activated protein C (PCa). It is in this form, PCa, that protein C, interacting with calcium ions as well as phospholipids and its cofactor, protein S, is capable of interacting with its substrates and in particular in the coagulation process, with factors Va and VIIIa (FVa, FVIIIa). Thus, the inhibition of factor Va can be assayed in order to measure the activity of the thrombomodulin.

Measurement of the functional activity of activated protein C comprises reaction of the reagent obtained, with an enzymatic substrate of activated protein C.

This substrate was described above; it will be recalled that it may be a natural or synthetic substrate. When it is a natural substrate, it may be a plasmatic coagulation protein, such as FVa or FVIIIa. Alternatively, the substrate measured, which reveals the functional activity of thrombomodulin, may be activated TAFI (TAFIa) or a substrate for activated TAFI. When the enzymatic substrate for PCa is synthetic in nature, it may, for example, be a peptide substrate. Advantageously, the substrate in question is a chromogenic or fluorometric substrate.

In particular, it may be a substrate which can assay the amidolytic activity of PCa. Such a substrate is, for example, the synthetic oligopeptide substrate CBS 42.46 from Diagnostica Stago. Other substrates have been cited above by way of example.

The step for quantification of the functional activity of thrombomodulin is based on quantification of the transformation of the substrate for PCa.

This quantification may be carried out by measuring the optical density of the reaction mixture in a reading window which is determined as a function of the selected substrate.

In accordance with a particular implementation of the invention, the plasma obtained from the biological sample taken from the patient is diluted. As an example, the plasma is diluted less than twenty times, for example less than ten times and in particular it is diluted to ⅓ or ¼.

The dilution which is carried out may be decided as a function of the sensitivity of the substrate: the more sensitive the substrate, the greater may be the dilution.

In a particular implementation of the invention, the fibrin polymerization inhibitor is a polypeptide inhibitor. In particular, it may be the polypeptide H-Gly-Pro-Arg-Pro-OH.AcOH (GPRP.AcOH) (Pefabloc® FG from Pentapharm). This inhibitor may be used in an assay reaction at a concentration of 1 to 15 g/l, selected as a function of the dilution of the plasma sample. As an example, when the plasma is diluted by ⅓, 10 g/l of this inhibitor is advantageously used.

In another implementation of the invention, the fibrin polymerization inhibitor is an anti-fibrinogenic antibody as described above.

In accordance with a particular implementation of the invention, measurement of the functional activity of the thrombomodulin comprises using a thrombin buffer with the following constitution:

NaCl (0.15 mmol/L [millimole/liter]), Tris (20 mmol/L), CaCl₂ (2.5 mmol/L), BSA (5 mg/ml) and
when the plasma sample derives from a patient being treated with heparin, a heparin inhibitor, for example polybrene (10 g/l).

Any other buffer which is suitable for diluting thrombin may be used.

When the enzymatic substrate for the activated protein C is CBS 42.46, or any other enzymatic substrate, in particular a synthetic substrate, the optical density may be read within a reading window of 6 to 500 seconds at a wavelength of 405 nm.

Alternatively, the optical density may be recorded within a window of 6 to 200 s, at a wavelength of 405 nm.

The concentration of substrate, in particular CBS 42.46, is, for example, 2.5 μM/ml in the reagent to be added to the reaction medium.

The step for incubating the plasma in contact with thrombin in the presence of calcium ions with the protein C may be carried out for a period of approximately 1 to 120 minutes, for example approximately 600 seconds.

Thus, in a particular implementation of the invention, the method for measuring the functional activity of PCa is carried out under the following conditions:
for step a), 70 μl of plasma sample diluted to ⅓ is brought into contact with 40 μl of 10 NIH thrombin diluted in a buffer containing NaCl (0.15 mmol/L), Tris (20 mmol/L), CaCl₂ (2.5 mmol/L), BSA (5 mg/ml) and in the presence of a fibrin polymerization inhibitor, for example GPRP.AcOH (10 g/l) and, when the plasma sample derives from a patient treated with heparin, a heparin inhibitor, for example polybrene (10 g/l);
for step b), 30 μl of purified protein C is incubated for 600 s with the medium constituted in step a);
for step c), 110 μl of enzymatic substrate for the activated protein C, for example CBS 42.46 in a concentration of 2.5 μM/ml (in the reagent) and the quantity of transformed enzymatic substrate is determined by reading the optical density within a period of 6 to 500 s, at a wavelength of 405 nm.

In a particular implementation of the invention, the functional activity of the plasmatic thrombomodulin is measured in parallel to the measurement of the same activity of an internal control. This control may be produced from a standard plasma treated in the same manner as the sample.

The quantities and/or concentrations of the reagents to be supplied to the reaction medium in order to carry out the method, like the quantities and/or concentrations of plasma, thrombin, purified protein C, fibrin polymerization inhibitor and substrate, may vary from the figures given in the present application, in particular as a function of the concentration of each reagent or its activity. Preferably, the respective final concentrations of the various reagents of the reaction are substantially conserved in the reaction medium.

The term “final concentration” means the concentration of reagents in the medium where they act when they have been introduced.

The skilled person can adapt the concentrations and activities as a function of the selected reagents. By way of illustration, it should be noted that the concentrations or activities of the reagents may vary by an amount of plus or minus 50%, for example by an amount of plus or minus 20% or by plus or minus 10% compared with the indications contained in the present application. This variation may concern each reagent, independently of the other reagents.

In order to quantify the functional activity of thrombomodulin, in accordance with a particular implementation, the invention proposes comparing the result obtained for the reaction, for example the recorded value for the optical density, with values drawn from a calibration curve. This calibration curve, for example, is obtained by measuring the functional activity of thrombomodulin by means of:
a standard plasma sample diluted in Owren Koller buffer, for example a plasma which comprises the usual statistical quantities, termed normal, of known coagulation factors in the endogenous and exogenous coagulation pathways. It may be obtained from normal plasma samples, or from pooled normal samples, or from individually tested samples in order then to determine a mean value for the individual values obtained for the thrombomodulin activity. Statistically normal values for the coagulation factors are situated in the ranges given in the manuals which are known to the skilled person. As an example, a normal plasma is considered to be a plasma giving results qualified as normal in fibrinogen tests, cephalin time activated (CTA), thrombin time (TT), PC and factor V. In this regard, reference may also be made to the standards defined in the work “How to define and determine reference intervals in the clinical laboratory: Approved Guidelines—Second Edition. NCCLS document C28-A2 (ISBN 1-56238-406-6) NCCLS-USA 2000”; or
a plasma depleted in thrombomodulin to which an increasing quantity of purified TM is added. It may be obtained using any method which is known per se, for example by immunoadsorption using an anti-thrombomodulin antibody, to carry out TM depletion before adding purified TM.

It is also possible to form a dilution range of normal plasma from a pool of normal plasma or plasma rendered deficient in TM to which increasing quantities of TM are added.

The thrombomodulin added to the plasma samples to establish the calibration curve may be complete or soluble.

It may also concern a truncated and soluble recombinant thrombomodulin molecule, such as Solulin® marketed by Berlex Biosciences, San Francisco, USA. This molecule may be used as a standard to produce an internal control or to produce a calibration curve.

Depending on the characteristics of the patients who may be subjected to the TM activity measurement test, the choice of plasma samples used to obtain the control values may be adjusted.

Thus, the plasma samples selected as controls will advantageously be taken from individuals with characteristics close to those of the test patients as regarding the age, sex, body mass index, and if appropriate level of tobacco or alcohol consumption.

The invention also pertains to a kit for measuring the activity of plasmatic thrombomodulin, in a plasmatic medium, comprising:
a reagent 1 comprising thrombin diluted in a buffer comprising a fibrin polymerization inhibitor and comprising calcium ions and, if appropriate, a heparin inhibitor;
a reagent 2 comprising purified protein C; or, alternatively, TAFI (thrombin activatable fibrinolysis inhibitor);
a reagent 3 comprising an enzymatic substrate for activated protein C or a specific substrate for TAFI.

Assaying TAFI activity consisting of measuring TAFIa (activated TAFI) has been described, for example, in patent WO-03/004516 using chromogenic substrates of TAFIa derived from arazoformyl compounds.

In accordance with a particular implementation of the invention, the kit is characterized in that reagent 1 essentially comprises the following compounds: NaCl (0.15 mmol/L), Tris (20 mmol/L), CaCl₂ (2.5 mmol/L), BSA (5 mg/ml) and for plasma samples from patients treated with heparin or another anticoagulant, a heparin or said anticoagulant inhibitor, for example polybrene (10 g/l), and a fibrin polymerization inhibitor.

In accordance with a particular implementation of the invention, the fibrin polymerization inhibitor is GPRP.AcOH used in a concentration of 1 to 15 g/l. The concentration is determined as a function of the degree of dilution of the test plasma. As an example, when the plasma is diluted by a third, 10 g/l of GPRP.AcOH is used. The properties and conditions for use of this inhibitor were described above.

The invention also pertains to a kit as defined above wherein the fibrin polymerization inhibitor is an anti-fibrinogen antibody.

In a particular implementation, the kit further comprises an internal control defined as indicated in the present application.

In accordance with a particular implementation, the kit may also comprise a calibration curve for the functional activity of thrombomodulin or means for producing such a curve.

The invention also pertains to a method for in vitro detection of a coagulation anomaly, comprising measuring the functional activity of thrombomodulin in a plasmatic medium, as described in the present application. It may concern detecting an increase in the level of the functional activity of the thrombomodulin or a reduction in that level.

In an adult subject, an abnormal variation (increase or decrease) in the thrombomodulin level is a marker for the risk of thrombosis or of endothelial lesion(s).

In accordance with a particular implementation of this method, the measured functional activity of the thrombomodulin in a plasmatic medium is compared with a value for this activity measured using a standard plasma.

In accordance with a particular implementation of the invention, the value obtained, termed the standard or normal value, is obtained after assaying the functional activity of thrombomodulin on a pool of standard plasma samples.

In accordance with a particular implementation of the invention, said standard value is established from values for the functional activity of thrombomodulin measured using normal plasma samples (standards) which are individually assayed.

The means, methods and kits described in the present application can be used to detect an increase in the thrombomodulin level, possibly associated with clinical symptoms. Thus, an increase in the thrombomodulin level may be associated with symptoms of disseminated intravascular coagulation (DIVC), diabetes, chronic myeloid leukemia, hepatic and renal insufficiency, preeclampsia, thrombotic thrombocytopenic purpura, rickettsioses, Behcet's disease, homocystinurea or miscarriage.

In accordance with another implementation of the invention, the means, methods and kits can be used to detect a reduction in the thrombomodulin level, possibly associated with clinical symptoms. These symptoms may be the symptoms of pulmonary arterial hypertension or melanoma or others.

In accordance with another implementation of the invention, the means, methods and kits described are used to detect a reduction in the level of thrombomodulin associated with a mutation in the gene for thrombomodulin.

Other characteristics and advantages of the invention will become apparent from the following examples, which in particular illustrate the variations in the thrombomodulin levels linked to pathological states, and from the figures.

FIG. 1: Diagram of the organizational structure of thrombomodulin: EGF=epidermal growth factor (EGF-like). The sequence for thrombomodulin is described in the article by Weiler H and Isermann B H in 2003 (Journal of Thrombosis and Haemostasis, vol 1, pages 1515-1524);

FIG. 2: Diagram of thrombomodulin activity: PC: protein C; PCa: activated protein C; Th: thrombin; ATIII-Th: anti-thrombin III-thrombin complex; EPCR-PC: endothelial protein C receptor-protein C; TAFI: Thrombin Activatable Fibrinolysis inhibitor; TAFIa: activated TAFI; TM: Thrombomodulin; V: Factor V; Va: activated Factor V;

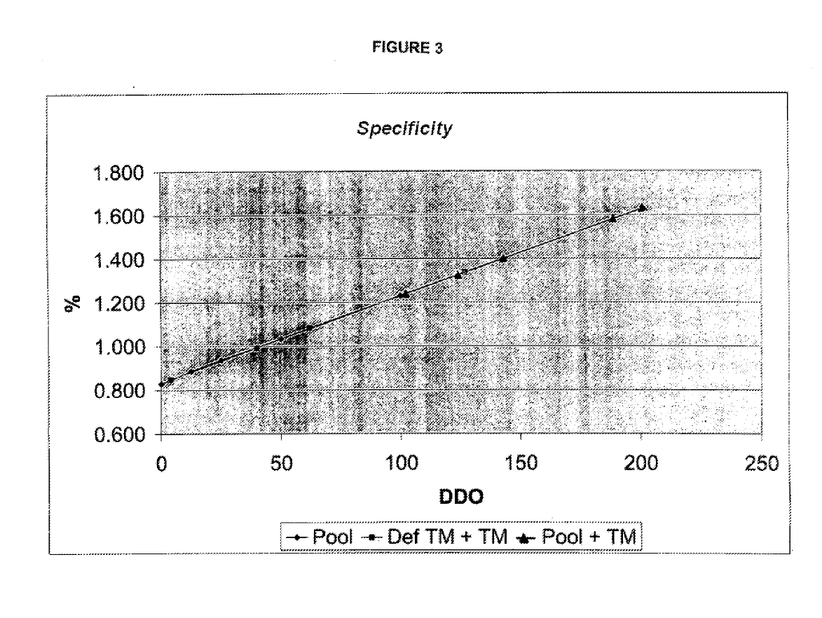

FIG. 3: Curve illustrating assay specificity (Def TM: deficient in TM);

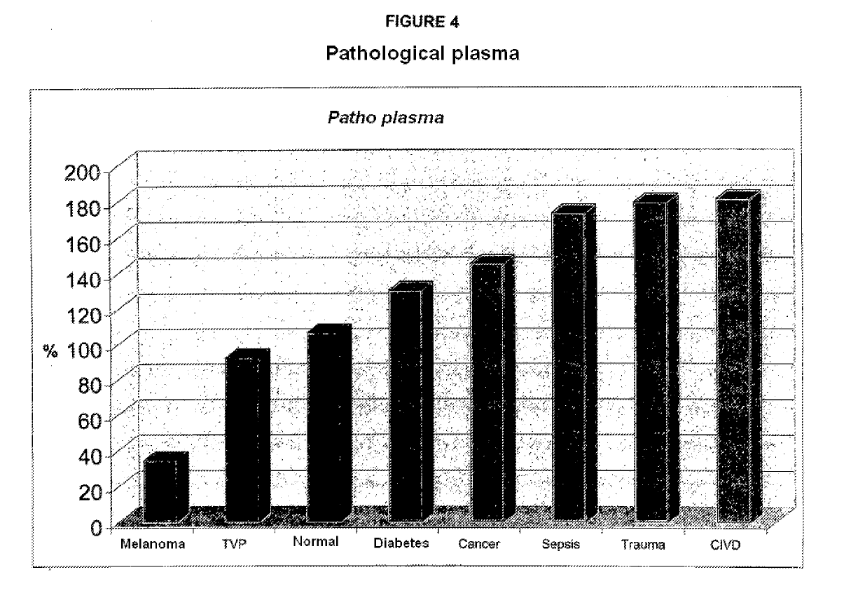

FIG. 4: Detection of pathological plasma and normal plasma;

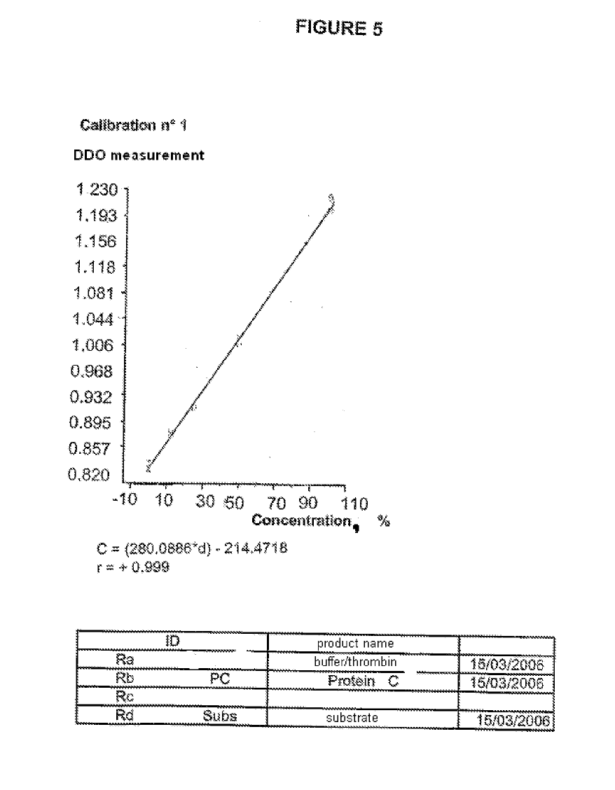

FIG. 5: Calibration curve for measurement (DDO) of thrombomodulin activity as a function of its concentration;

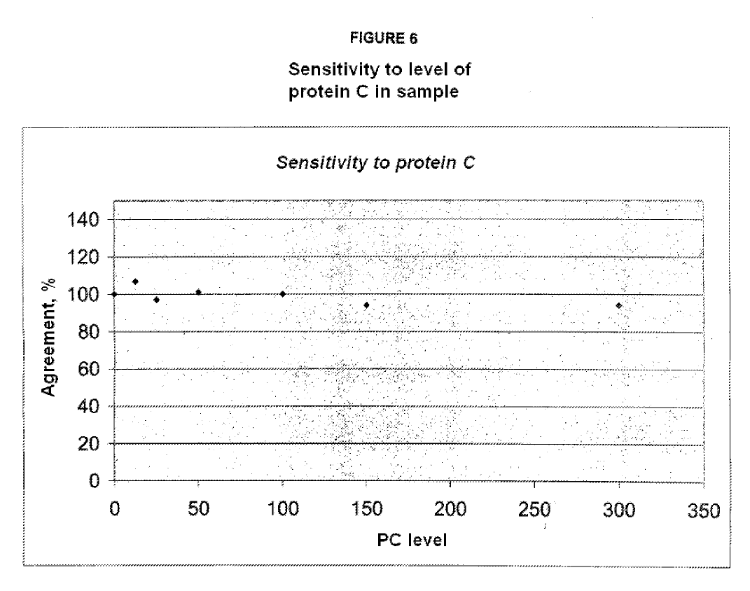

FIG. 6: Measurement of sensitivity of thrombomodulin activity as a function of the amount of protein C in the sample;

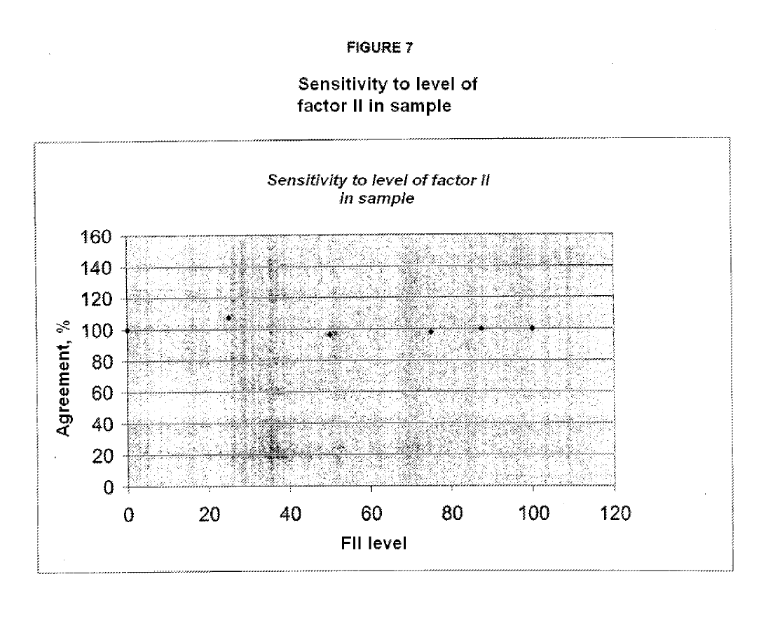

FIG. 7: Sensitivity of factor II of sample;

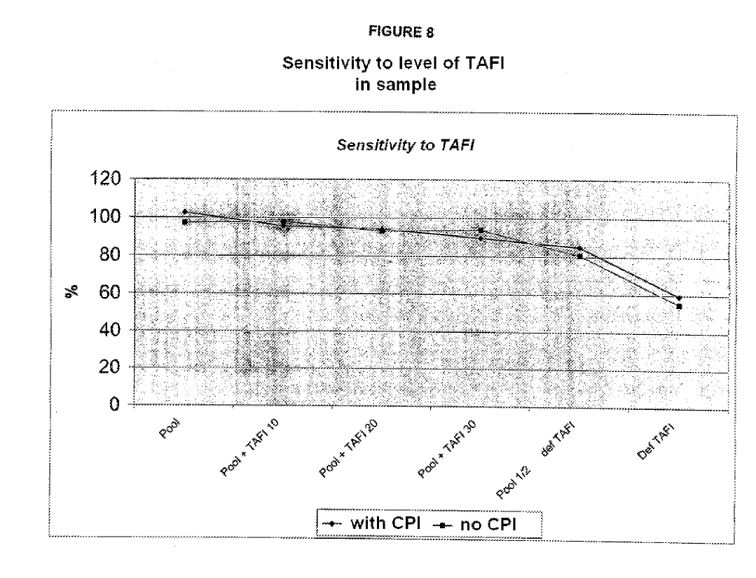

FIG. 8: Sensitivity of amount of TAR in sample;

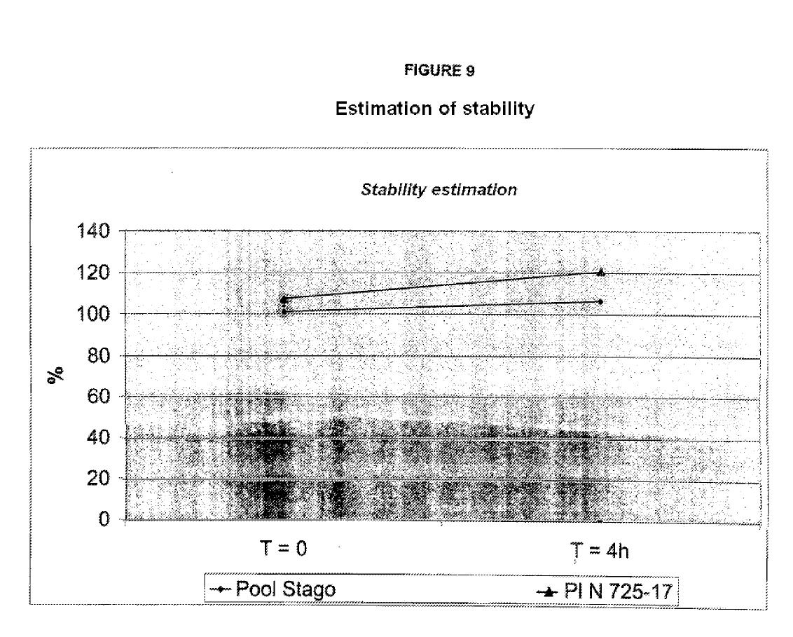

FIG. 9: Estimation of stability;

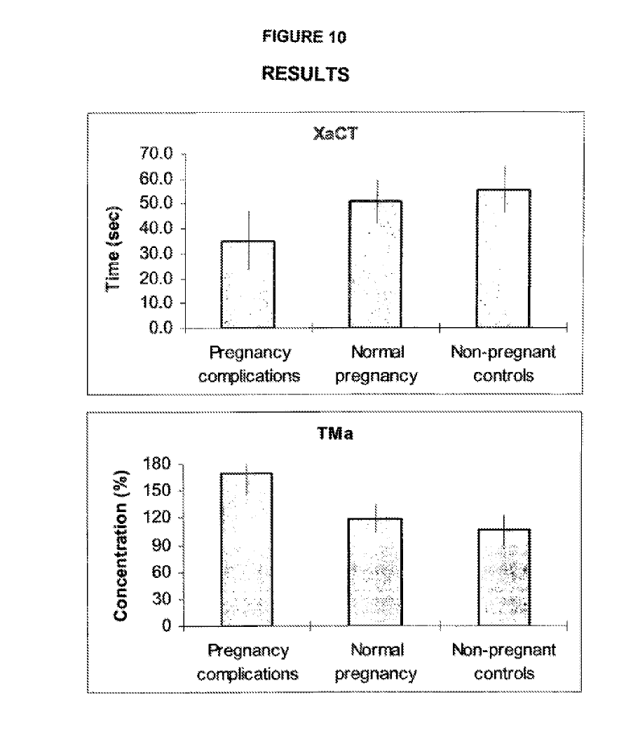

FIG. 10: Measurement of concentration of phospholipids and TMa activity for plasma samples from women with normal pregnancies or suffering from complications;

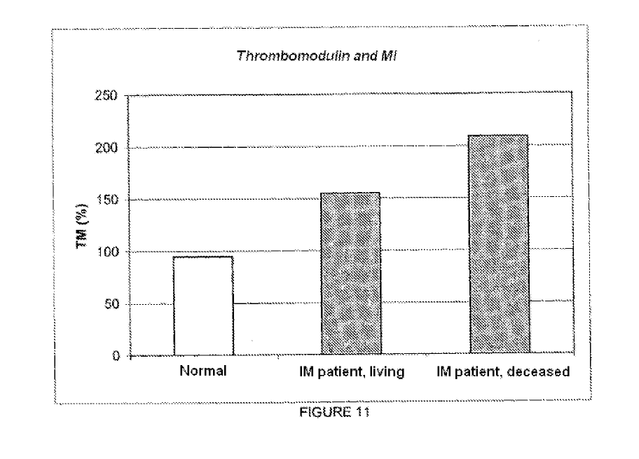

FIG. 11: Activity of thrombomodulin correlated with myocardial infarctus (MI);

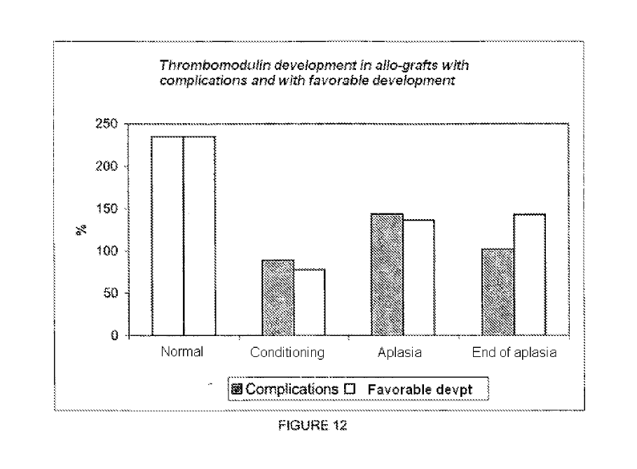

FIG. 12: change in thrombomodulin from patients who have undergone an allo-graft. The TMa activity was tested on samples from patients whose condition changed to one with complications and on samples from patients whose condition changed favorably.

The conditions for producing these results are described in the examples.

### EXAMPLES

### 1. Assay Method

The thrombomodulin functional activity was assayed as follows on a 70 μl plasma sample diluted ⅓.

### A) Reagents to be Supplied to Reaction Medium

40 μl of 10 NIH thrombin (i.e. 1.6 NIH final in the medium) in thrombin buffer constituted by:
0.15 mmol/L NaCl
20 mmol/L Tris
2.5 mmol/L CaCl₂
5 mg/ml BSA
10 g/l GPRP.AcOH (fibrin polymerization inhibitor)
2 ml/l polybrene, 10 g/l
30 μl of purified protein C, 100 μg/ml, taken up in 1 ml of distilled water
110 μl of synthetic substrate for PCa (CBS 42.46 from Diagnostica Stago, 2.5 μM/ml) taken up in 6 ml of distilled water/hirudin, 20 ATU) (i.e. final concentration in medium of 1.10 μM/ml).

### B) Activation of Protein C and Assay of PCa

The plasma sample was brought into contact with thrombin in the thrombin buffer and incubated at 37° C. with protein C, for a period of approximately 600 seconds, to activate the protein C.

The medium obtained was brought into contact with the synthetic substrate.

The amidolytic activity of the PCa was then determined at 37° C. by reading the optical density in a reading window of 6 to 500 sec, at 405 nm

### C) Checking Specificity of Measurement

The specificity of the measurement of the optical density for the PCa activity was verified by comparing the activity obtained with a pool of normal plasma and with a plasma deficient in TM to which purified TM had been added (from American Diagnostica or Haematologic Technologies Inc, for example). The results are shown in FIG. 3 and confirm the specificity of the assay.

### D) Checking Repeatability

The repeatability measurements were carried out and the results are shown in Tables 1-3.

### E) Values for Activity of PCa

43 normal plasma were assayed for thrombomodulin activity, using the method of the invention. The optical density measurements and the corresponding TM activity percentages are shown in Table 4. These measurements could be considered to be indicators of the normal values for functional activity of thrombomodulin.

TABLE 4
Normal plasma

Normal (n = 43)

| | DDO | % |
|---|---|---|
| Mean | 1.281 | 106 |
| Median | 1.291 | 109 |
| Min | 1.122 | 65 |
| Max | 1.451 | 151 |

100% TM functional activity was considered to be arbitrarily defined as the amount of TM which results in the formation of 0.03 μmol/l of PCa when the concentrations of thrombin and PC are respectively 10 NIH and 100 μg/ml in the presence of 2.5 mmol/L of Ca²⁺ ions.

Further, plasma obtained from biological samples taken from patients suffering from identified pathologies were also assayed for the TM functional activity using the method of the invention. The measurements were carried out in a STA-R analyzer (Diagnostica Stago, France) using the chromogenic test with the synthetic substrate mentioned above. The intra and inter test Cts values were respectively <4% and <5%.

The response to the test was linear between 0 and 200%. The results as a percentage of TM activity were as follows: diabetes (n=15): 130%±28; cancers (n=10): 155±60; sepsis (n=12): 173%±56; multiple trauma (n=20): 185%±62.

These results are shown in FIG. 4.

### II Example of Preparation of a Calibration Curve

In order to produce the calibration curve giving the functional activity of thrombomodulin, the following was used:
either dilution of a pool of normal plasma in Owren Koller buffer or in a thrombomodulin deficient;
or overloading a thrombomodulin-deficient plasma with purified thrombomodulin;
or by arbitrarily defining the level of 100% of thrombomodulin activity as being the thrombomodulin level which results in the formation of 0.03 μml/l of PCa when the concentration of thrombin is 10 NIH and the concentration of PC is 100 μg/ml, in the presence of 2.5 mmol/L of Ca²⁺.

Each plasma dilution was tested at ⅓ in the configuration.

The concentration of TM added to the plasma pool corresponding to an activity of 200%, 150%, 100%, 50%, 25%, 12.5% and 0% of the thrombomodulin was determined using the rule defined above.

The activity of the thrombomodulin was determined on a synthetic substrate of PCa, CBS 42.46. The amidolytic activity of the PCa was measured by para-nitroaniline liberation (yellow color) at a wavelength of 405 nm.

The optical density which corresponded to each value (as a percentage) of thrombomodulin activity was determined in a reading window of 6 to 500 seconds at a wavelength of 405 nm.

A calibration curve is shown in FIG. 5.

### III Evaluation of the Sensitivity of Assay to Various Plasmatic Coagulation Proteins or to Agents Interfering with Coagulation

### A) Sensitivity to Heparin

The sensitivity was determined by adding increasing concentrations of Calciparine® to the assayed plasma under the assay conditions described above.

The influence of calciparine is described in Table 6 below and was shown to be of little effect on the functional activity of TM.

TABLE 6
Sensitivity to heparin

| | DDO | % | Agreement |
|---|---|---|---|
| Calciparine 0 | 1.183 | 119 | 100 |
| Calciparine 0.4 | 1.135 | 107 | 90 |
| Calciparine 0.8 | 1.159 | 113 | 95 |
| Calciparine 1.0 | 1.199 | 123 | 103 |
| Calciparine 1.4 | 1.239 | 133 | 111 |
| Calciparine 1.8 | 1.271 | 141 | 118 |
| Calciparine 2.0 | 1.214 | 127 | 106 |

### B) Sensitivity to Level of Protein C Contained in Plasma Sample

Plasma comprising a predetermined variable concentration of protein C was assayed using the method described above.

It was as follows:
plasma deficient in protein C (column 0);
plasma from a pool rendered deficient in protein C to varying extents (columns 12.5-50);
plasma from a normal pool (GK (Georges King) pool) (column 100).

The results (Table 7 and FIG. 6) show that the level of PC in the sample has little influence on the value of the functional activity of TM.

TABLE 7
Sensitivity to amount of protein C in sample (1)

| | Level of PC (%) | | | | | | |
|---|---|---|---|---|---|---|---|
| | 0 | 12.5 | 25 | 50 | 100 | 150 | 300 |
| Theoretical | 110 | 103 | 104 | 106 | 102 | 102 | 102 |
| level (%) | | | | | | | |
| Measured | 110 | 110 | 101 | 107 | 102 | 96 | 96 |
| level (%) | | | | | | | |
| Agreement | 100 | 107 | 97 | 101 | 100 | 94 | 94 |

0 = Def PC Clinisys
12.5-50 = Pool/Def PC
100 = GK pool
150-300 = Pool/purified PC.

### C) Sensitivity to Level of Factor II Contained in Plasma Sample

Plasma containing a predetermined variable concentration of factor II were assayed using the method described above.

This was as follows:
plasma deficient in factor II (column 0);
plasma from a pool rendered deficient in factor II to varying extents (columns 25-87.5);
plasma from a normal pool (GK pool) (column 100).

The results (Table 8 and FIG. 7) show that the level of FII of the sample has little influence on the value of the functional activity of TM.

TABLE 8

Level of FII (%)

| | 100 | 87.5 | 75 | 50 | 25 | 0 |
|---|---|---|---|---|---|---|
| Theoretical | 100 | 95 | 90 | 79 | 69 | 58 |
| level (%) | | | | | | |
| Measured | 100 | 95 | 88 | 76 | 74 | 58 |
| level (%) | | | | | | |
| Agreement | 100 | 100 | 98 | 96 | 107 | 100 |

0 = Def II GK
25-87.5 = Pool/Def II
100 = GK pool

### D) Sensitivity to Level of TAFI Contained in Plasma Sample

Plasma comprising a predetermined variable concentration of TAFI was assayed using the method described above.

This was as follows:
plasma deficient in TAFI;
plasma from a pool rendered deficient in TAFI to varying extents;
plasma from a normal pool (GK pool).

The results (Table 9 and FIG. 8) show that the level of TAFI in the sample has little influence on the value of the functional activity of the TM.

TABLE 9
Sensitivity to level of TAFI of sample (1)

| | | Pool + | Pool + | Pool + | | Pool ½ | |
|---|---|---|---|---|---|---|---|
| | | TAFI | TAFI | TAFI | | def | Def |
| Samples | | 10 | 20 | 30 | Pool | TAFI | TAFI |
| With | DDO | 1.102 | 1.097 | 1.079 | 1.144 | 1.077 | 0.980 |
| CPI | % | 95 | 94 | 89 | 103 | 85 | 59 |
| Without | DDO | 1.112 | 1.094 | 1.097 | 1.124 | 1.062 | 0.964 |
| CPI | % | 98 | 93 | 94 | 97 | 81 | 55 |

| | | | | | | |
|---|---|---|---|---|---|---|
| Δ | −3 | 1 | −5 | 5 | 4 | 4 |

### E) Estimation of Stability

The assay of the functional activity of TM was reproduced after a time interval in order to estimate the stability of the reaction. The assay carried out at 4 h intervals on the same sample showed that the values had the correct stability (Table 10 and FIG. 9).

TABLE 10

| | T = 0 | | T = 4 h | | Relative |
|---|---|---|---|---|---|
| | DDO | % | DDO | % | deviation |
| Stago pool | 1.195 | 101 | 1.216 | 106 | 5 |
| PIN 725-17 | 1.220 | 107 | 1.274 | 121 | 13 |

PIN 725-17 was a normal plasma supplied by Manchester and corresponding to batch 725-17.

### F) Reading Windows

The reading window for the optical densities was adjusted in order to record in a window of 6-500 seconds then a window of 6-200 seconds.

The results obtained for the various plasma samples were substantially equivalent (Table 11).

### IV Correlation Between an Increase in Thrombomodulin Activity and the Early Loss of a Foetus in a Pregnant Woman

Early foetal loss is a serious clinical problem which cannot always be adequately explained or detected. Apart from anatomical, chromosomal or hormonal causes, anti-phospholipid syndrome (APS) is the single common cause which can be diagnosed up to now. One of the mechanisms proposed to explained miscarriage is hypoxia due to utero-placental thrombosis. Thrombomodulin activity may be an important marker in this thrombosis. A test in accordance with the invention for thrombomodulin activity based on its capacity to promote activation of protein C by thrombin, was carried out in a control study in patients who had suffered early miscarriage. The results obtained from blood samples collected during the first trimester of pregnancy in patients (N=35) who had no history of thrombosis and who had suffered an early miscarriage (not linked to APS) were compared with the results obtained from samples obtained during the first trimester of pregnancy in women of a similar age group with a normal pregnancy (N=37) and with normal patients who were not pregnant (N=32). The blood samples were collected from the group of patients immediately after miscarriage. All of the blood samples (1:10 in 109 MN of citrate) were stirred two times and the plasma was frozen at −80° C. until the test for aPTT and for thrombomodulin activity (Diagnostica Stago, France). No modification in the aPTT time was observed. The mean values for the thrombomodulin activity test were 106±12% for the control as opposed to 120±10% for normal pregnancies and 158±14% for the miscarriages (p less than 0.001). Although supplemental studies would be required to determine the capacity and specificity of this test, determination of this parameter may prove to be a useful tool in assisting in detecting women at risk of a miscarriage. The thrombomodulin activity appears to be an advantageous early predictive marker of hypertensive complications in pregnancy which, in a woman at risk, could allow pharmaceutical intervention intended to prevent more severe clinical manifestations.

TABLE 11

| | Normal sample | Normal pregnancy | Miscarriage |
|---|---|---|---|
| | n = 32 | n = 37 | n = 35 |
| TM | 106 | 120 | 158 |
| (activity) | 65-139 | 69-152 | 77-279 |
| aPTT | 34.7 | 33.9 | 32.7 |
| | 29.4-39.6 | 29.5-40.4 | 28.6-37.3 |

### V Correlation Between Thrombomodulin Activity and Complications in Pregnancy

Complications in pregnancy involve the activity of NK (natural killer) cells and utero-placental thrombosis.

Procoagulant phospholipids (PPL) may modulate the role of NK activity and TMa increases in the case of vascular damage.

Measurements of the concentration of PPLs using a coagulation test based on factor Xa (XaCT—Xa clotting time) and measurement of the TMa activity may be of interest since these tests are predictive markers for the detection of women who are susceptible to experiencing complications in pregnancy.

Blood samples from women (35) without a history of thrombosis who had encountered complications during pregnancy (not linked to antiphospholipid syndrome), in women (37) of the same age group with a normal pregnancy and in women (32) with no identified pathology and who were not pregnant were tested. The samples were treated to collect the plasma which was stored at −80° C. prior to the test.

The PPLs were measured in a XaCT test. A shortened coagulation time reflected a rise in PPL concentration. The TMa activity was measured in a chromogenic test based on the capacity to promote activation of protein C (as described above).

A comparison of the results obtained (FIG. 10) on the 3 types of samples showed: 1) for XaCT tests: means of 35.2±11.8 s; 50.6±8.6 s and 55±9.2 s respectively for patients who had a pregnancy with complications, a normal pregnancy, and the controls (p<0.001); 2) for the TMa activity tests: means of 169%±24, 119%±16 and 106%±16 respectively for pregnancies with complications, normal pregnancies and controls (p<0.05).

It thus appears that both the XaCT tests and the TMa are of interest as predictive markers in the detection of women who are at risk of complications in their pregnancy.

### VI Correlation Between an Increase in Thrombomodulin Activity and Myocardial Infarctus

Thrombomodulin activity was measured for a pool of normal plasma and a plasma sample taken from a living patient suffering from mycocardial infarctus (MI) and from a blood sample taken from a patient who had died following an MI.

The measurement was carried out under the conditions described at point I above.

The results show a substantial increase in the thrombomodulin activity in the case of MI.

| | TM % |
|---|---|
| Normal | 95 |
| Living MI patient | 155 |
| Deceased MI patient | 209 |

### VII Correlation Between Thrombomodulin Activity and Marrow Allo-Graft

Thrombomodulin activity was measured for plasma samples taken from patients (children) who had undergone a bone marrow allo-graft.

The TM activity after the graft varied depending on the stage at which the sample was taken following the graft. A difference was observed in the change in the TM activity depending on whether the graft developed with complications or, in contrast, favorably.

1-35. (canceled)

36. A method for in vitro measurement of the functional activity of thrombomodulin, said method comprising assaying, in a biological medium from a biological sample, activation of protein C to activated protein C (PCa) by thrombin in the presence of its cofactor, said thrombomodulin, said method comprising adding to the plasma sample the agents necessary for activation of the protein C system, adding purified protein C and also adding a fibrin polymerization inhibitor.

37. The in vitro thrombomodulin functional activity measurement method as claimed in claim 36, wherein the assayed thrombomodulin is plasmatic thrombomodulin, the biological sample being a plasma sample, the assay being carried out in a plasmatic medium.

38. The method as claimed in claim 36, wherein in order to activate the protein C system, thrombin and calcium ions are added.

39. The method as claimed in claim 36, wherein the activity of the activated protein C is determined using an enzymatic substrate.

40. The method as claimed in claim 39, wherein the amidolytic activity of the activated protein C is assayed using a chromogenic or fluorometric substrate for PCa.

41. The method as claimed in claim 40, wherein the liberation of para-nitroaniline by the synthetic substrate CBS 42.46 is assayed.

42. The method as claimed in claim 39, wherein a plasmatic protein of coagulation is assayed the inhibition of which depends on PCa, for example factor Va, factor VIIIa or TAFIa.

43. The method as claimed in claim 37, comprising the steps of:
a) bringing a plasma sample into contact with a reagent 1 comprising thrombin, contained in a medium which can activate the protein C system, in particular comprising calcium ions, the reagent 1 further comprising a fibrin polymerization inhibitor;
b) incubating the medium constituted in step a) with a reagent 2 comprising purified protein C, for a period which is sufficient to allow its activation to PCa by the thrombin complexed to its cofactor, namely the thrombomodulin contained in the plasma sample;
c) bringing the medium constituted in step b) comprising the activated protein C into contact with an enzymatic substrate for PCa;
d) detecting the transformation of the substrate for the PCa.

44. The method as claimed in claim 37, wherein the plasma sample is diluted, for example diluted by ⅓.

45. The method as claimed in claim 36, wherein the fibrin polymerization inhibitor is a polypeptide inhibitor, for example H-GlyProArgPro-OH.AcOH (SEQ ID NO: 1) (GPRP.AcOH).

46. The method as claimed in claim 36, wherein the fibrin polymerization inhibitor is an anti-fibrinogen antibody.

47. The method as claimed in claim 43, wherein the thrombin buffer essentially comprises the following compounds:
NaCl (0.15 mmol/L), Tris (20 mmol/L), CaCl₂ (2.5 mmol/L), BSA (5 mg/ml); and
when the plasma sample is characteristic of a patient treated with heparin, a heparin inhibitor, for example polybrene (10 g/l).

48. The method as claimed in claim 45, wherein the fibrin polymerization inhibitor, GPRP.AcOH (SEQ ID NO: 1), is added in a concentration of 1 to 15 g/l depending on the dilution of the plasma sample.

49. The method as claimed in claim 43, wherein the enzymatic substrate for the activated protein C is the oligopeptide THC-Pro-Arg-pNa.

50. The method as claimed in claim 39, wherein the quantitative measurement for the transformation of the enzymatic substrate for the PCa is carried out by recording the optical density.

51. The method as claimed in claim 43, wherein the constituents employed in the reaction are used under the following conditions:
for step a), 70 μl of plasma sample diluted to ⅓ is brought into contact with 40 μl of 10 NIH thrombin diluted in a buffer containing NaCl (0.15 mmol/L), Tris (20 mmol/L), CaCl₂ (2.5 mmol/L), BSA (5 mg/ml) and in the presence of a fibrin polymerization inhibitor, for example GPRP.AcOH (SEQ ID NO: 1) (10 g/l) and, when the plasma sample derives from a patient treated with heparin, a heparin inhibitor, for example polybrene (10 g/l);
for step b), 30 μl of purified protein C is incubated for 600 seconds with the medium constituted in step a);
for step c), 110 μl of enzymatic substrate for the activated protein C, the quantity of transformed enzymatic substrate being determined by reading the optical density within a period of 6 to 500 s, at a wavelength of 405 nm.

52. The method as claimed in claim 36, characterized in that it further comprises assaying an internal control.

53. The method as claimed in claim 36, wherein the values obtained for the optical density are compared with a calibration curve.

54. The method as claimed in claim 43, wherein the reagent is thrombin contained in a thrombomodulin-deficient plasma, to which has been added a fibrin polymerization inhibitor and, when the test plasma is characteristic of a patient treated with an anticoagulant, an inhibitor of said anticoagulant.

55. A kit for functionally measuring thrombomodulin activity in a biological medium, especially a plasmatic medium, comprising:
a reagent 1 comprising thrombin diluted in a buffer comprising a fibrin polymerization inhibitor and comprising calcium ions;
a reagent 2 comprising purified protein C;
if appropriate, a reagent 3 comprising an enzymatic substrate for activated protein C.

56. A kit as claimed in claim 55, wherein the reagent 1 essentially comprises the following compounds: NaCl (0.15 mmol/L), Tris (20 mmol/L), CaCl₂ (2.5 mmol/L), BSA (5 mg/ml) and when the plasma sample derives from a patient treated with heparin, a heparin inhibitor, for example polybrene (10 g/l), and a fibrin polymerization inhibitor.

57. The kit as claimed in claim 55, wherein the fibrin polymerization inhibitor is GPRP.AcOH (SEQ ID NO: 1) used in a concentration of 1 to 15 g/l.

58. A kit for functionally measuring thrombomodulin activity in a biological medium, especially a plasmatic medium, comprising:
a reagent 1 comprising thrombin diluted in a buffer comprising a fibrin polymerization inhibitor and comprising calcium;
a reagent 2 comprising TAFI; and, if appropriate
a reagent 3 comprising a substrate for TAFI.

59. The kit as claimed in claim 55, wherein it further comprises an internal control.

60. The kit as claimed in claim 54, wherein it comprises a calibration curve for the functional activity of thrombomodulin.

61. A method for in vitro detection of a coagulation anomaly, comprising measuring the functional activity of plasmatic thrombomodulin as claimed in claim 37.

62. The method as claimed in claim 60, comprising comparing the level of functional activity measured for thrombomodulin with a normal value for said activity.

63. The method as claimed in claim 62, wherein the normal value is obtained after assaying the functional activity of the thrombomodulin for a pool of normal plasma samples.

64. The method as claimed in claim 62, wherein the normal value is established from values for the functional activity of thrombomodulin measured on individually assayed normal plasma samples.

65. The method as claimed in claim 36, for the detection of an increase in the level of thrombomodulin associated with symptoms of disseminated intravascular coagulation (DIVC), diabetes, chronic myeloid leukaemia, hepatic and renal insufficiency, preeclampsia, thrombotic thrombocytopenic purpura, rickettsioses, Behcet's disease, homocystinuria, and miscarriage.

66. The method as claimed in claim 36, for the detection of a reduction in the level of thrombomodulin associated with symptoms of pulmonary arterial hypertension or melanoma.

67. The method as claimed in claim 36, for the detection of a reduction in the level of thrombomodulin associated with a mutation in the gene for thrombomodulin.

68. The kit as claimed in claim 55, for the detection of an increase in the thrombomodulin level associated with symptoms of disseminated intravascular coagulation (DIVC), diabetes, chronic myeloid leukaemia, hepatic and renal insufficiency, preeclampsia, thrombotic thrombocytopenic purpura, rickettsioses, Behcet's disease, homocystinuria, and miscarriage.

69. The kit as claimed in claim 55, for the detection of a reduction in the level of thrombomodulin associated with symptoms of pulmonary arterial hypertension or melanoma.

70. The kit as claimed in claim 55, for the detection of a reduction in the level of thrombomodulin associated with a mutation in the gene for thrombomodulin.


SEQUENCE LISTING (1 sequence)

SEQ ID NO: 1 - protein, 4 aa, Artificial Sequence
  Synthetic Oligopeptide
GPRP